(19) United States
(12) Patent Application Publication
(10) Pub. No.: US 2008/0267467 A1
(43) Pub. Date: Oct. 30, 2008

(54) METHOD AND SYSTEM FOR AUTOMATIC ADJUSTMENT OF A DIAGNOSTIC IMAGING DISPLAY

(75) Inventors: Alexander Sokulin, Kiriat Tivon (IL); Ran Menirom, Haifa (IL); Doron Hess, Haifa (IL); Meir Aizen, Haifa (IL)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/742,300

(22) Filed: Apr. 30, 2007

(51) Int. Cl. G06K 9/00; A61B 8/00

(52) U.S. Cl. 382/128; 600/437

(57) ABSTRACT

A method and system for automatic adjustment of a diagnostic imaging display are provided. The system includes an acquisition component configured to acquire image data and a display configured to display the acquired image data. The diagnostic imaging system further includes an ambient light detector configured to detect an ambient light level and a display adjustment module configured to automatically adjust a display transfer function for the display based on the detected ambient light level.

### BACKGROUND OF THE INVENTION

This invention relates generally to diagnostic imaging systems, and more particularly, to automatically adjusting display settings of a display of the diagnostic imaging system.

Diagnostic imaging systems, and in particular, medical imaging systems are used to image patients under many different conditions. For example, a medical imaging scanner may be used to perform imaging in different rooms having different lighting conditions, such as sun lit or bright lights in one room and dim lights or dark in another room. The surrounding ambient light affects the image displayed on the screen of the medical imaging system. As medical imaging systems continue to become more portable or mobile, the conditions under which the systems will operate will change more frequently, for example, when moving the mobile systems from one room or location to another room or location.

Users often neglect to readjust the screen settings to allow for proper and optimal viewing. This failure to readjust may be because the user forgot to adjust the screen or does not want to take the time to manually adjust the settings. The manual adjustment process can take time because a user usually will view a display displaying some typical images or a special test pattern containing some known fixed gray levels and adjust the screen using these images or pattern.

Medical imaging systems are also increasingly using screens other than traditional cathode ray tubes (CRTs) to display medical images. For example, plasma displays screens or liquid crystal display (LCD) screens are increasingly used. The affects of variable ambient light conditions are particularly apparent when using LCD screens because of the lower dynamic range of the LCD screens. Accordingly, LCD screens are less tolerant to changes in ambient lighting than CRT screens. Thus, adjustment of the LCD screen is more important and requires more precise changes to the screen settings. Moreover, performing manual adjustment often will not provide an optimal image, which may result in improper diagnosis because an object in an image may not be visible. Additionally, in these LCD screens, the display transfer functions (e.g., gamma curve functions) for the screens, which are used to achieve a correct reproduction of luminance for optimal viewing, are often stored in look up tables. Accordingly, unlike CRT screens that all typically have the same transfer function, LCD screens may have variations in the transfer function between different manufacturers and models. This variation in transfer functions may cause an image to be displayed acceptably on one LCD screen, but unacceptably on another LCD screen. An image will also appear different when viewed on an LCD screen versus a CRT screen.

Moreover, if the display screen of the medical imaging system is not correctly adjusted, for example, not balanced correctly for the current ambient light, users often compensate for the incorrectly adjusted displayed image, particularly if the image is gray-scale, by adjusting the level of total gain of the displayed image. This method of manual adjustment often results in a sub-optimal signal-to-noise ratio. Additionally, stored image data containing any improper compensation will produce an image that can appear unbalanced when viewed at a later time on a well adjusted display screen.

In some situations, lighting conditions in a room may change while viewing the display screen. For example, if the display screen is adjusted for a dark room and the ambient light conditions increase (e.g., a window allows more light into the room as clouds clear from the sky), in some cases, data containing diagnostic information (e.g., low level echo data) may become completely obscured (e.g., become black) if the display screen is not readjusted. In these situations, particularly when the light increase is gradual, the user may not be aware that relevant medical data is being lost from view because of the changing light conditions. Improper diagnosis may result.

Additionally, depending on the type of image, improper screen adjustment may have even greater adverse affects. For example, ultrasound images may have the most common gray levels that are typically the 20-30 darkest levels. Accordingly, ultrasound images are typically different than standard digital images (e.g., digital camera pictures) that are displayed on a screen. Commercial monitors and screens are manufactured for use to display many different types of images having different characteristics and properties that may be displayed by office users or home users. However, because these screens are typically optimized for displaying images over a vast display range, ultrasound images are usually not displayed optimally, and may be displayed below an acceptable viewing level, particularly because of the typically dark gray levels often present in these types of images.

### BRIEF DESCRIPTION OF THE INVENTION

In accordance with an embodiment, a diagnostic imaging system is provided that includes an acquisition component configured to acquire image data and a display configured to display the acquired image data. The diagnostic imaging system further includes an ambient light detector configured to detect an ambient light level and a display adjustment module configured to automatically adjust a display transfer function for the display based on the detected ambient light level.

In accordance with another embodiment, a medical imaging system is provided that includes a display configured to display medical images, a user interface configured to receive user inputs and an ambient light detector configured to detect ambient light in proximity to the display. The medical imaging system further includes a display adjustment module configured to automatically adjust settings of the display based on a detected ambient light level.

In accordance with yet another embodiment, a method for controlling a display of a diagnostic imaging system is provided. The method includes receiving ambient light level information and modifying a transfer function for the display based on the ambient light level information to satisfy an optimal display setting for the display.

### BRIEF DESCRIPTION OF THE DRAWINGS

FIG. 1 is a block diagram of a diagnostic imaging system constructed in accordance with an embodiment of the present invention.

FIG. 2 is a block diagram of an ultrasound imaging system constructed in accordance with an embodiment of the invention.

FIG. 3 is a top plan view of a user interface constructed in accordance with an embodiment of the present invention.

FIG. 4 is a diagram illustrating an ambient light detector constructed in accordance with an embodiment of the invention.

FIG. 5 is a diagram illustrating an ambient light detector constructed in accordance with another embodiment of the invention.

FIG. 6 is a diagram illustrating an ambient light detector constructed in accordance with another embodiment of the invention.

FIG. 7 is a perspective view of a portable medical imaging system constructed in accordance with an embodiment of the invention.

FIG. 8 is a perspective view of a hand carried medical imaging system constructed in accordance with another embodiment of the invention.

FIG. 9 is a perspective view of a pocket-sized medical imaging system constructed in accordance with another embodiment of the invention.

FIG. 10 is a block diagram illustrating display compensation performed in accordance with various embodiments of the invention.

FIG. 11 is graphs illustrating an ambient light compensation function and a screen type compensation function in accordance with an embodiment of the invention.

FIG. 12 is a graph illustrating a screen type compensation function in accordance with another embodiment of the invention.

FIG. 13 is a flow chart of a method for adjusting the settings of a display in accordance with various embodiments of the invention.

FIG. 14 is a graph of an ambient light compensation function in accordance with an embodiment of the invention.

FIG. 15 is a graph of a shifted ambient light compensation function in accordance with an embodiment of the invention.

FIG. 16 is a graph of a shifted ambient light compensation function in accordance with another embodiment of the invention.

FIG. 17 is a block diagram illustrating the calculation of a compensation function in accordance with an embodiment of the invention.

### DETAILED DESCRIPTION OF THE INVENTION

The foregoing summary, as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like). Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

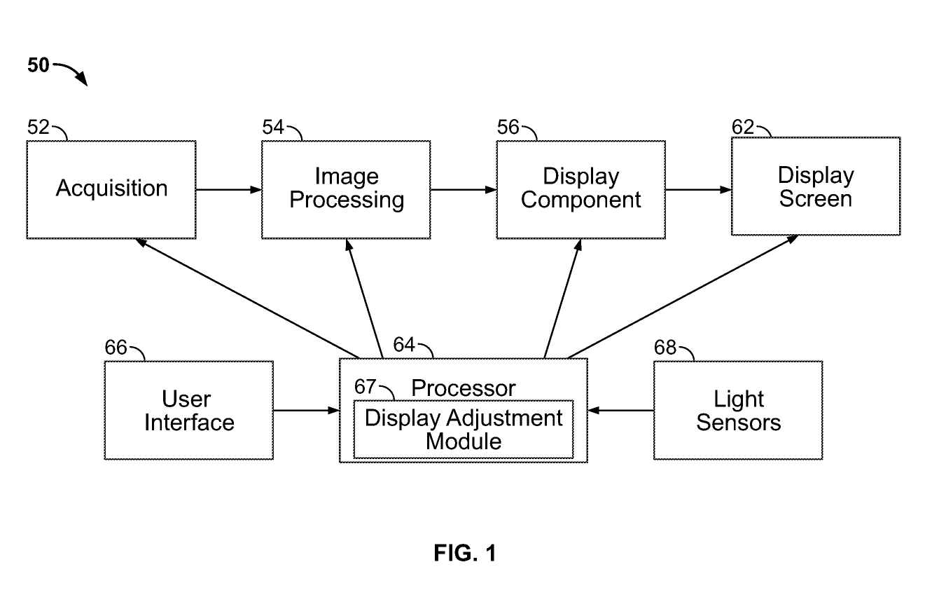

Various embodiments of the invention provide a diagnostic imaging system 50 as shown in FIG. 1 that automatically optimizes viewing of images. The diagnostic imaging system 50 may be any type of system, for example, different types of medical imaging systems, such as an ultrasound imaging system, an x-ray imaging system, a computed-tomography (CT) imaging system, a single photon emission computed tomography (SPECT) system, a positron emission tomography (PET) imaging system, a nuclear medicine imaging system, a magnetic resonance imaging (MRI) system, and combinations thereof (e.g., a multi-modality imaging system), among others. However, the various embodiment are not limited to medical imaging systems or imaging systems for imaging human subjects, but may include non-medical systems for imaging non-human objects and for performing non-destructive imaging or testing, security imaging (e.g., airport security screening), etc.

The diagnostic imaging system 50 generally includes an acquisition component 52 configured to acquire image data (e.g., ultrasound image data). The acquisition component 52 may be, for example, a probe, scanner or other similar device for scanning an object or volume of interest. The acquisition component 52 is connected to an image processing component 54. The image processing component 54 is any type of image processor capable of processing the acquired image data and is connected to a display component 56. The display component 56, which may be a controller, receives display correction information, such as a correction function calculated or determined by a display adjustment module 67 and configures or formats the processed image data for display on a display screen 62 as described in more detail herein. The display screen 62 may be any type of screen capable of displaying images, graphics, text, etc. For example, the display screen 62 may be a cathode ray tube (CRT) screen, a liquid crystal display (LCD) screen or a plasma screen, among others.

A processor 64 (e.g., computer) or other processing unit controls the various operations within the diagnostic imaging system 50. For example, the processor 64 may receive user inputs from a user interface 66 and display requested image data or adjust the settings for the displayed image data. For example, a user may provide manual brightness or contrast adjustment settings that are translated by the display adjustment module 67 (which may use one or more display look up tables) to change the display properties of the display screen 62. The processor 64 is also connected to one or more light sensors 68 (e.g., photocells) that provide information about the ambient lighting conditions of the area or room in which the diagnostic imaging system 50 is located and as described in more detail below. Using this ambient light information, the processor 64 uses the display adjustment module 67 to automatically adjust the settings (e.g., brightness and contrast) of the display screen 62.

Thus, in operation, the display screen 62 settings may be adjusted manually by a user or automatically based on measured ambient light conditions. As described in more detail herein, various embodiments of the invention use screen type compensation in combination with ambient light compensation to adjust the display screen 62. For example, based on the ambient lighting conditions the transfer function for the display screen is modified, which in some embodiments includes shifting a transfer curve for the particular transfer function for the display screen.

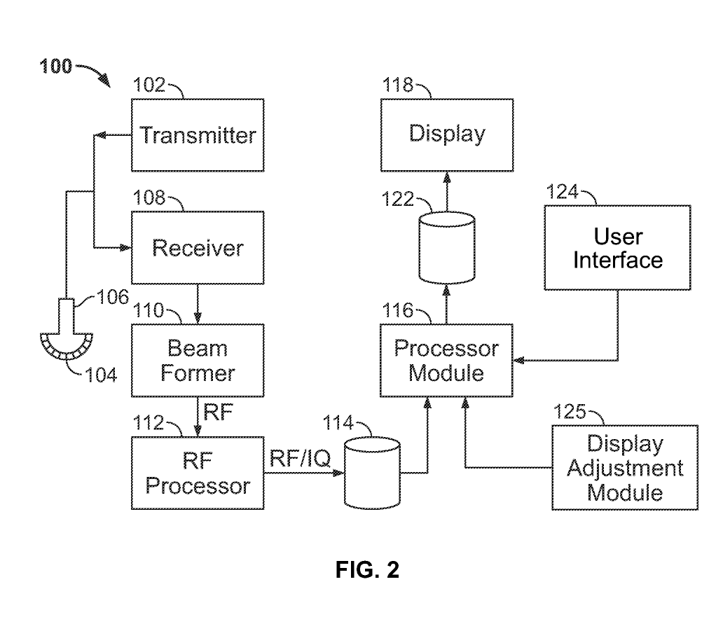

The diagnostic imaging system 50 may be, for example, an ultrasound system 100 shown in FIG. 2. The ultrasound system 100 includes a transmitter 102 that drives an array of elements 104 (e.g., piezoelectric elements) within a transducer 106 to emit pulsed ultrasonic signals into a body. A variety of geometries may be used. The ultrasonic signals are back-scattered from structures in the body, like blood cells or muscular tissue, to produce echoes that return to the elements 104. The echoes are received by a receiver 108. The received echoes are passed through a beamformer 110, which performs beamforming and outputs an RF signal. The RF signal then passes through an RF processor 112. Alternatively, the RF processor 112 may include a complex demodulator (not shown) that demodulates the RF signal to form IQ data pairs representative of the echo signals. The RF or IQ signal data may then be routed directly to a memory 114 for storage.

The ultrasound system 100 also includes a processor module 116 to process the acquired ultrasound information (e.g., RF signal data or IQ data pairs) and prepare frames of ultrasound information for display on display 118. The processor module 116 is adapted to perform one or more processing operations according to a plurality of selectable ultrasound modalities on the acquired ultrasound information. Acquired ultrasound information may be processed and displayed in real-time during a scanning session as the echo signals are received. Additionally or alternatively, the ultrasound information may be stored temporarily in memory 114 during a scanning session and the processed and displayed in off-line operation.

The processor module 116 is connected to a user interface 124 that may control operation of the processor module 116 as explained below in more detail. The display 118 includes one or more monitors that present patient information, including diagnostic ultrasound images to the user for diagnosis and analysis. The display 118 automatically adjusts luminance settings based on predetermined transfer functions that are shifted based on measured ambient light conditions. One or both of memory 114 and memory 122 may store three-dimensional data sets of the ultrasound data, where such 3-D data sets are accessed to present 2-D and 3-D images. The images may be modified and the display settings of the display 118 also manually adjusted using the user interface 124.

The ultrasound system 100 also includes a display adjustment module 125 (which may be the same as the display adjustment module 67 of FIG. 1) that is used to automatically adjust the display settings of the display 118. The display adjustment module 125 is configured to calculate or determine a display correction function used to perform automatic adjustment of the display, which may include screen type compensation and ambient light compensation as described in more detail below.

The system 100 may obtain volumetric data sets by various techniques (e.g., 3D scanning, real-time 3D imaging, volume scanning, 2D scanning with transducers having positioning sensors, freehand scanning using a Voxel correlation technique, 2D or matrix array transducers and the like). The transducer 106 is moved, such as along a linear or arcuate path, while scanning a region of interest (ROI). At each linear or arcuate position, the transducer 106 obtains scan planes that are stored in the memory 114.

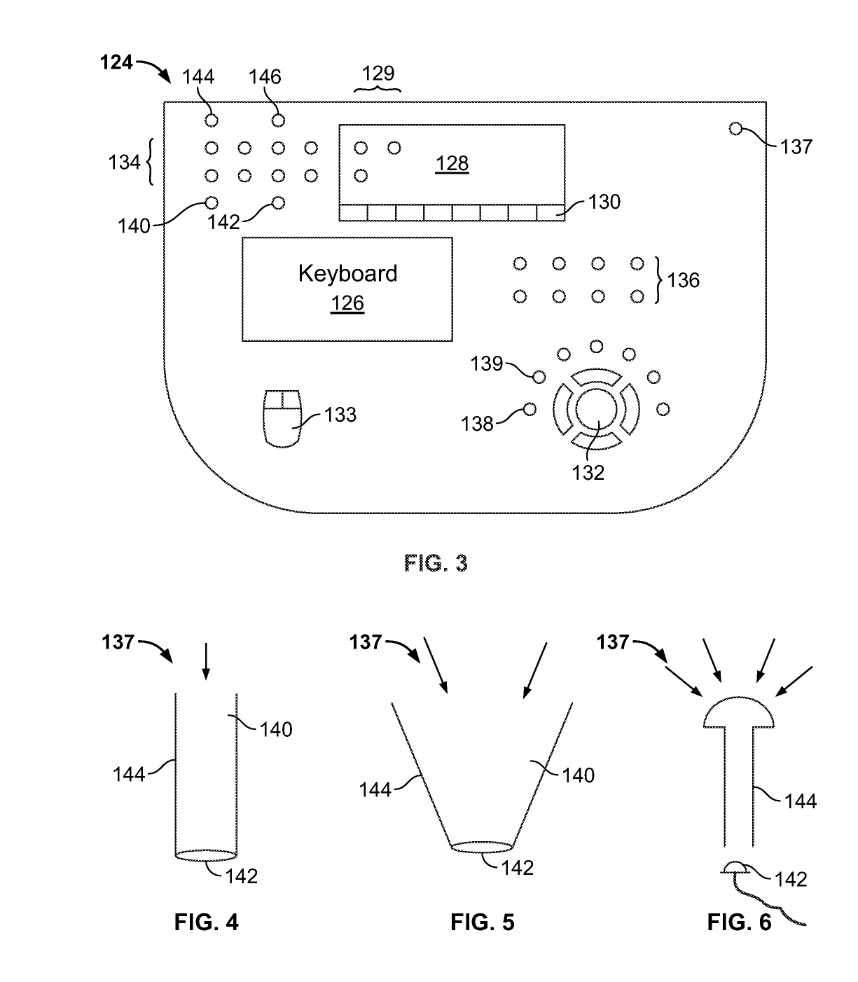

FIG. 3 illustrates the user interface 124 constructed in accordance with one embodiment of the invention. The user interface 124 includes a keyboard 126, a mouse 133, a touch screen 128, a series of soft keys 130 proximate the touch screen 128, a trackball 132, view position buttons 134, mode buttons 136 and control or operation keys 138. The soft keys 126 are assigned different functions on the touch screen 128 depending upon a selected examination mode, stage of examination and the like. The trackball 132 and keys 138 are used to control the display of images on the display 124 and control various options, for example, zoom, rotate, viewing mode, examination mode, etc. For example, the view position buttons 134 may change different views of the displayed image. Optionally, the view position buttons 134 may be implemented as touch areas 129 on the touch screen 128. As a further option, the size, position and orientation of the displayed image may be controlled partially or entirely by touch areas provided on the touch screen 128 and/or by the soft keys 130.

The user interface 124 also includes other controls, such as a save command/option 140 and a restore command/option 142 to save or restore certain image characteristics or changes to the displayed image. However, it should be noted that the various controls may be used to adjust or control different settings, display options, etc. For example, the user interface 124 may include a brightness control button 144 that allows a user to manually adjust screen brightness and a contrast control button 146 that allows a user to manually adjust screen contrast. For example, the brightness control button 144 may be used to enter a brightness control mode that allows a user to increase or decrease the brightness of the display 118 (shown in FIG. 2) using the touch areas 129 that may display up and down arrows to indicate brightness increase and brightness decrease, respectively. The contrast control button 146 likewise may be used to enter a contrast control mode that allows a user to increase or decrease the contrast of the display 118, again using the touch areas, where the arrows now increase and decrease screen contrast. The increasing or decreasing of the setting alternatively may be provided using other controls, such a moving the trackball 132 up/down or left/right. Any suitable controls may be provided to adjust the brightness or contrast, such as, roller wheels, dedicated toggles or buttons, etc.

The user interface 124 also includes an ambient light detector 137, for example, having a photocell assembly or similar device capable of measuring the level of ambient light. The ambient light detector 137 may be positioned at any location on the user interface 124. The ambient light detector 137 also may be provided on the display 118 and as described in more detail below. More than one ambient light detector 137 also may be provided. The ambient light detector 137, as shown in FIGS. 4 through 6, generally includes an opening 140 through which light (illustrated by arrows) may pass. The opening 140 is generally located on the surface of the user interface 124 or display 118 (e.g., a hole in a keyboard) and may be covered by a transparent covering (not shown). The opening 140 exposes a light level detector 142 (e.g., photocell) to the light via a light guide 144 (e.g., plastic rod or bar). The light guide 142 may be configured differently, for example, generally forming a cylindrical passage as shown in FIG. 4, a conical passage as shown in FIG. 5 or a curved upper end as shown in FIG. 6. The walls of the light guide 142 also may be made reflective to direct light toward the photocell 142. The walls of the light guide 142 also may be shaped or angled as desired or needed to optimize light measurements using the photocell. Thus, ambient light may be measured by the ambient light detector 137.

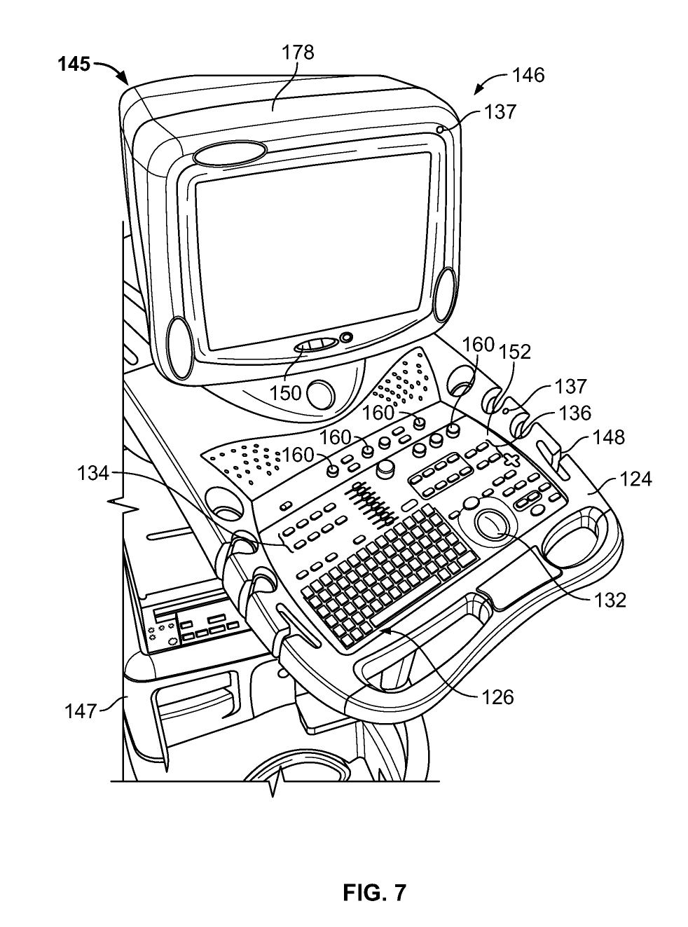

The ambient light detector 137 may be provided in connection with different imaging systems. For example, as shown in FIG. 7, the ambient light detector may be implemented in a portable imaging system 145 (e.g., portable ultrasound system) provided on a movable base 147. As shown, an ambient light detector 137 is provided on a top corner 146 of the display 118 and on a side 148 of the user interface 124. Various embodiments of the invention may use light from one or both of the ambient light detectors 137 to automatically control the luminance of the display 118. It should be noted that in the embodiment shown in FIG. 7, manual screen adjustment controls 150 (e.g., brightness and contrast controls) are provided on the display 118. It should be understood that the display 118 may be separate or separable from the user interface 124. The user interface 124 may optionally be a touchscreen, allowing the user to select options by touching displayed graphics, icons, and the like.

The user interface 124 of FIG. 7 also includes other optional control buttons 152 that may be used to control the portable imaging system 145 as desired or needed, and/or as typically provided. The user interface 124 provides multiple interface options that the user may physically manipulate to interact with ultrasound data and other data that may be displayed, as well as to input information and set and change scanning parameters. The interface options may be used for specific inputs, programmable inputs, contextual inputs, and the like. Different types of physical controls are provided as different physical actions are more intuitive to the user for accomplishing specific system actions and thus achieving specific system responses.

For example, multi-function controls 160 are positioned proximate to the display 118 and provide a plurality of different physical states. For example, a single multi-function control may provide movement functionality of a clockwise/counterclockwise (CW/CCW) rotary, up/down toggle, left/right toggle, other positional toggle, and on/off or pushbutton, thus allowing a plurality of different states, such as eight or twelve different states. Different combinations are possible and are not limited to those discussed herein. Optionally, less than eight states may be provided, such as CW/CCW rotary functionality with at least two toggle positions, such as up/down toggle and/or left/right toggle. Optionally, at least two toggle positions may be provided with pushbutton functionality. The multi-function controls 160 may be configured, for example, as joystick rotary controls.

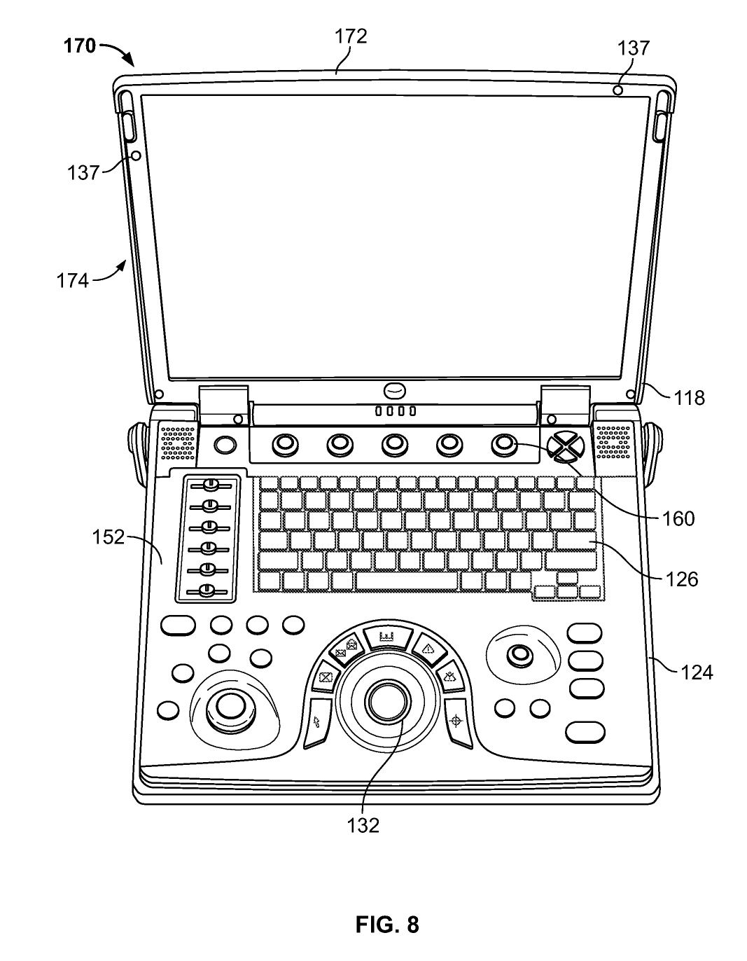

The ambient light detector 137 also may be provided in connection with a hand carried imaging system 170 as shown in FIG. 8 wherein the display 118 and user interface 124 form a single unit. The hand carried imaging system 170 may be, for example, a handheld or hand carried ultrasound imaging device, such as a miniaturized ultrasound system. As used herein, “miniaturized” means that the ultrasound system is a handheld or hand carried device or is configured to be carried in a person's hand, pocket, briefcase-sized case, or backpack. For example, the hand carried imaging system 170 may be a hand carried device having a size of a typical laptop computer, for instance, having dimensions of approximately 2.5 inches in depth, approximately 14 inches in width, and approximately 12 inches in height. The hand carried imaging system 170 may weigh about ten pounds.

The ambient light detector 137 may be provided at one or more locations on the hand carried imaging system 170. For example, an ambient light detector 137 may be provided on each of a top corner of the display 118. One or more ambient light detectors 137 optionally or alternatively may be provided along an outer edge 172 of the display 118, on a back side 174 of the display, on the user interface 124, for example, adjacent the keyboard 126 or may replace one of the various buttons or controls on the user interface 124.

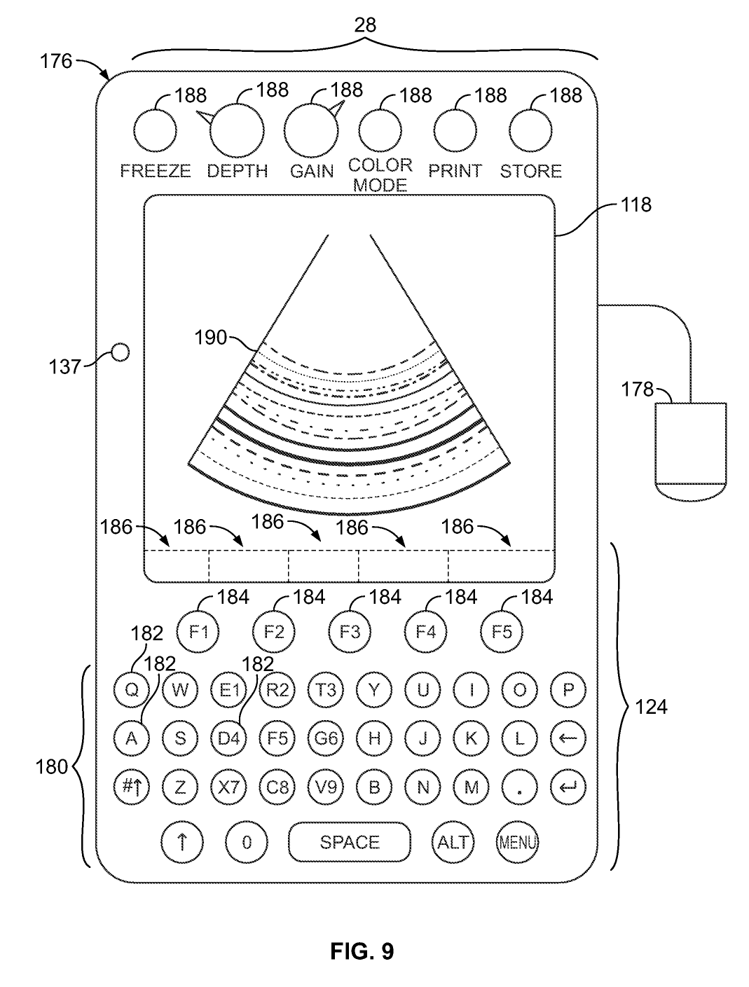

The ambient light detector 137 also may be provided in connection with a pocket-sized imaging system 176 as shown in FIG. 9 wherein the display 118 and user interface 124 form a single hand held unit. By way of example, the pocket-sized imaging system 176 may be a pocket-sized or hand-sized ultrasound system approximately 2 inches wide, approximately 4 inches in length, and approximately 0.5 inches in depth and weigh less than 3 ounces. The pocket-sized imaging system 176 generally includes the display 118, user interface 124, which may include a keyboard and an input/output (I/O) port for connection to a scanning device, for example, an ultrasound probe 178. The display 118 may be, for example, a 320×320 pixel color LCD display (on which a medical image 190 may be displayed). A typewriter-like keyboard 180 of buttons 182 may be included in the user interface 124. Multi-function controls 184 may each be assigned functions in accordance with the mode of system operation as previously discussed. As each of the multi-function controls 184 may be configured to provide a plurality of different physical actions, the mapping of system response to intuitive physical action may be improved without requiring additional space. Label display areas 186 associated with the multi-function controls 184 may be included as necessary on the display 118. The device may also have additional keys and/or controls 188 for special purpose functions, which may include, but are not limited to “freeze,” “depth control,” “gain control,” “color-mode,” “print,” and “store.”

One or more ambient light detectors 137 are provided, for example, adjacent the display 118. For example, ambient light detectors 137 may be provided proximate a side of the display 118 such that ambient light is measured in close proximity to the image 190 being displayed.

It should be noted that the various embodiments may be implemented in connection with miniaturized imaging systems having different dimensions, weights, and power consumption. In some embodiments, the pocket-sized ultrasound system may provide the same functionality as the system 100 (shown in FIG. 1).

It also should be noted that the size and shape of the ambient light detector 137 may be modified. For example, the opening 140 (shown in FIGS. 4 through 6) may be square or triangular. Also, and for example, a plurality of ambient light detectors 137 may be provided, at least some of which have different sizes and/or shapes. The shape or size of the ambient light detector 137 may be based, for example, on the positioning of the ambient light detectors 137. Also, the ambient light detectors 137 may be a separate unit that attaches to an imaging system. For example, the ambient light detector 137 may be a separate module that attaches to a top of a user interface or that may be retracted or extend from the user interface.

Various embodiments of the invention automatically control the settings of a display, for example, the display screen 62 of the diagnostic imaging system 50 (shown in FIG. 1) or the display 118 of the ultrasound system 100 (shown in FIG. 2). Using one or more ambient light detectors 137, display parameters, including, for example, brightness and contrast are dynamically adjusted as a function of surrounding ambient light as measured by the ambient light detector 137.

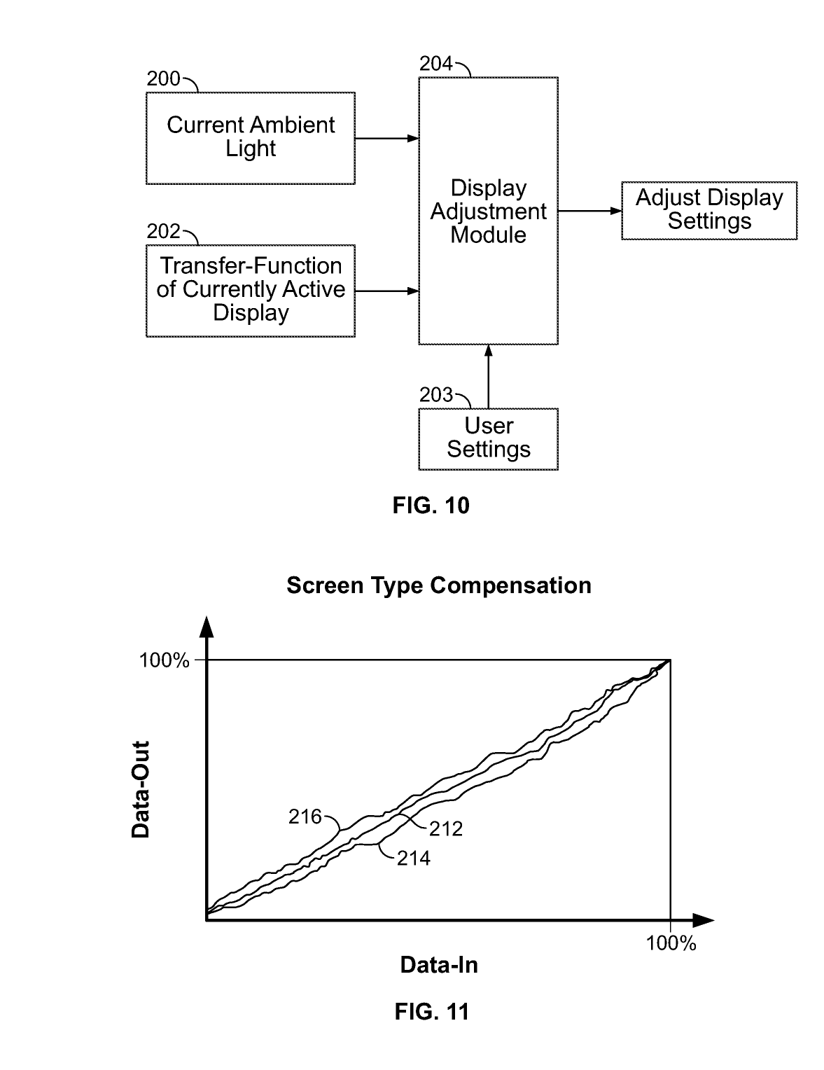

In particular, as shown in FIG. 10, current ambient light information 200 and transfer function information 202 (including a pre-defined or predetermined standard) of the currently active display are used to provide display compensation using a display adjustment module 204 (which may be the same as the display adjustment modules 67 and 125 of FIGS. 1 and 2, respectively), which may include screen type compensation and ambient light compensation. Optionally, user settings (e.g., manual settings or special settings) also may be used to provide display compensation 204. The determined compensation is then used to adjust display settings. For example, using the current measured ambient light and the screen transfer function (which may be based on measurement, technology, manufacturer, model, etc.) the display screen is automatically adjusted, or alternatively, adjusted upon a user request. The adjustment includes, but is not limited to:
1. Optimizing the dynamic range of a gray-scale image by setting or adjusting the brightness and contrast settings to compensate for ambient light conditions.
2. Converging the transfer function of the display screen to a pre-defined or predetermined standard (which be referred to as the optimal standard or gold standard).
3. Optimizing the color-gamut to display colors as expected by a user (e.g., adjust to natural color look as viewed by a user).

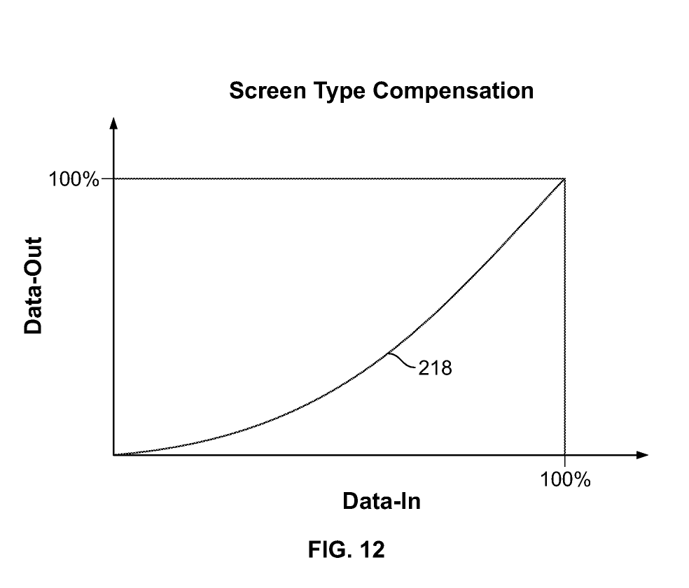

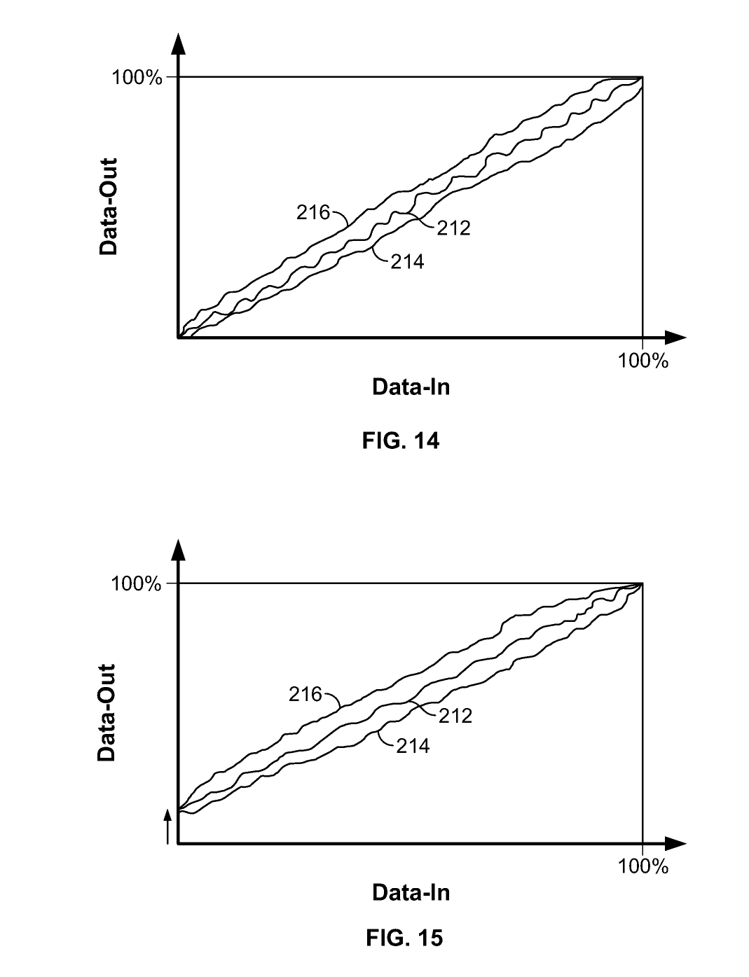

The various embodiments provide an optimized display look up table by combining ambient light compensation and screen type compensation. For example, as shown in FIG. 11, a screen type compensation function is defined by curves 212, 214 and 216 (representing red color, green color and blue color functions, respectively) and which may be combined with ambient light compensation to define functions represented by a curve 250 as shown in FIGS. 14 through 16. It should be noted that the functions are not necessarily linear, for example, as shown in FIG. 12, the transfer function may be represented by a non-linear curve 218.

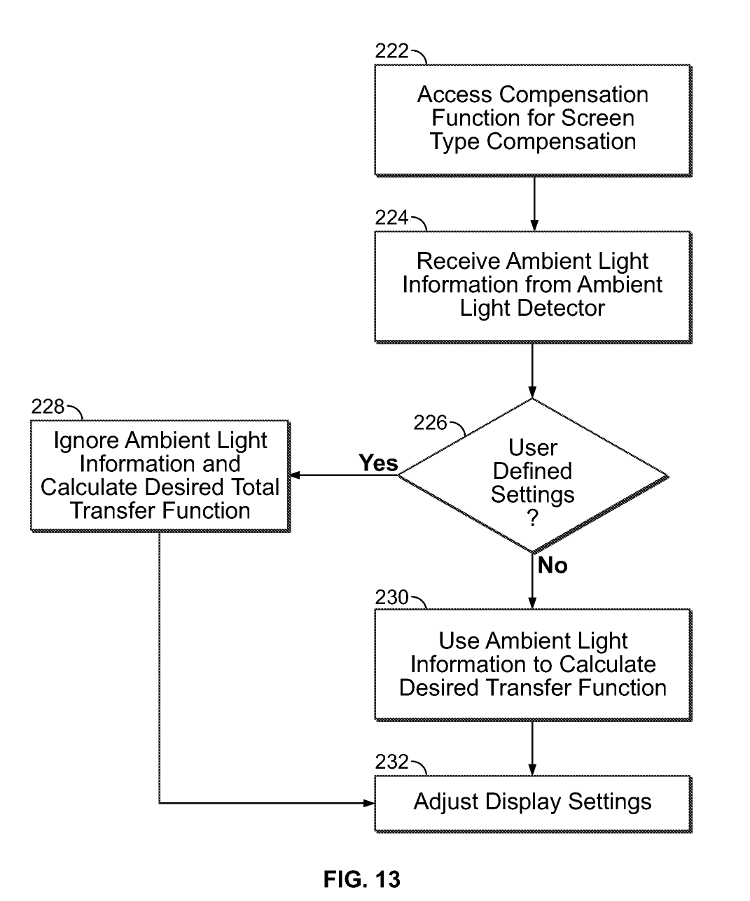

More particularly, various embodiments of the invention provide a method 220 as shown in FIG. 13 for adjusting the settings of a display, for example, a monitor or screen of a diagnostic imaging system, such as a medical imaging system. The method 220 includes accessing at 222 a compensation function, and more particularly, a screen type compensation function for the active screen. The compensation function may be stored in a look up table and is based on the real transfer function for the particular display. As used herein, transfer function refers to any function that may be used to correct or compensate for screen settings, including, but not limited to brightness, contrast and color. It should be noted that the real transfer function may be based on measurements performed on the particular display or calculated based on manufacturer or model information, etc. For example, a display color analyzer, such as a Konica Minolta CA-210 may be used to measure the white balance, white uniformity and luminance (with color values) of the display (and for each different type of display). A real transfer function for each type, brand, model, etc. of display may thereby be measured and a compensation function calculated or determined according to the following:
Total Transfer Function is y(x)=f(F(x)) with the optimal standard or gold standard defined as:

y(x)=g(x) (1)

Accordingly, g(x)=f(F(x)) and the correction function is defined as follows:

F(x)=f⁻¹(g(x)) (2)
where f(t) is the measured screen transfer function and g(x) is the optimal standard or gold standard transfer function.
   Thus, as shown in FIG. 17, an input x is applied to a correction function 260, t=F(x) with the output of the correction function over time t applied to a screen transfer function 262, y=f(t), which then provides the screen output y. It should be noted that the compensation function may provide separate compensation for each of the red, green and blue colors of the display as described herein.

It should be noted that the various embodiments may be implemented in connection with different types of imaging system, for example, different types of diagnostic imaging systems. The optimal settings or gold standard may be based on, for example, evaluations of users experienced in viewing these types of displays or particular types of images on the displays. The display settings for these users may be combined, averaged or otherwise used to establish the gray scale and color transfer functions that define the optimal settings or gold standard. A separate pre-defined optimal or gold standard display or transfer function may be provided, for example, for each of a plurality of imaging modalities. The pre-defined optimal settings or gold standard may be used to converge individual displays to an optimal display (which may be within predetermined tolerances).

Referring again to FIG. 13, once the compensation function is accessed, a current ambient light level is determined, and more particularly, at 224 ambient light information (e.g., current ambient light level) is received from an ambient light detector. This may include a determination of whether the ambient light to which the diagnostic imaging device is exposed has changed. This change may occur, for example, because the lighting in the room in which the diagnostic imaging device is located has changed or the diagnostic imaging device may have been moved to another location in the room with different lighting or may have been moved to another room (e.g., from a room with a window to a room without a window). The determination may be made using any type of light sensor or light detecting device, for example, the ambient light detector 137 or the lights sensors 68. For example, a photocell may be used to determine a current light level (which also may be compared to a previously measured light level). The photocell may be, for example, a photodiode having an output current proportional to the light intensity. It should be noted that the light level may be measured from one or more sensors or detectors. In such a case, the value from the sensor with the maximum value (e.g., highest brightness) may be used or alternatively the sensor values may be averaged. Also, signals from the sensors or detectors may be filtered (e.g., apply an adaptive filter) to filter out noisy events, for example, short or small events that affect the light level, such as, if a user accidentally covers one of the sensors or detectors.

A determination is then made at 226 as to whether a user input has been provided, for example, if user defined screen settings have been received. For example, a user may manually adjust the brightness or contrast settings of the screen. As another example, a user may have predefined stored settings for the display that are recognized when the user logs into the diagnostic imaging system (e.g., based on a username). If user defined setting have been received, then at 228 the ambient light information received at 224 is ignored and a desired total transfer function is calculated as described herein and that is based on the optimal standard or gold standard for the particular display. The total transfer function is modified (e.g., shifted and/or pivoted) based on the user input. If user defined settings have not been received, then at 230 the ambient light information is used and the desired total transfer function calculated as described herein, which is based on the optimal standard or gold standard for the particular display. For example, an automatic correction mode may be selected automatically or manually by a user.

The display is then adjusted at 232 based on the calculated total transfer function. This includes adjusting the settings of the display based on the calculated total transfer function that has been shifted and/or pivoted. It should be noted that a combination of user defined settings and ambient light information may be used. For example, the user defined settings may determine initial settings for the display with subsequent adjustments based on changes in ambient light conditions. A user may then also modify these settings. For example, if an initial user setting (e.g., initial manual setting) is provided for the display at a particular ambient light condition, then in one embodiment the ambient light compensation, and in particular, the corrections are made relative to the initial user setting, thereby maintaining the contrast and brightness properties initially set by a user. A prompt may be displayed indicating that user defined settings are being used.

The method 220 may be repeated periodically, for example, based on time intervals or upon detecting a changed condition (e.g., change in ambient light), etc.

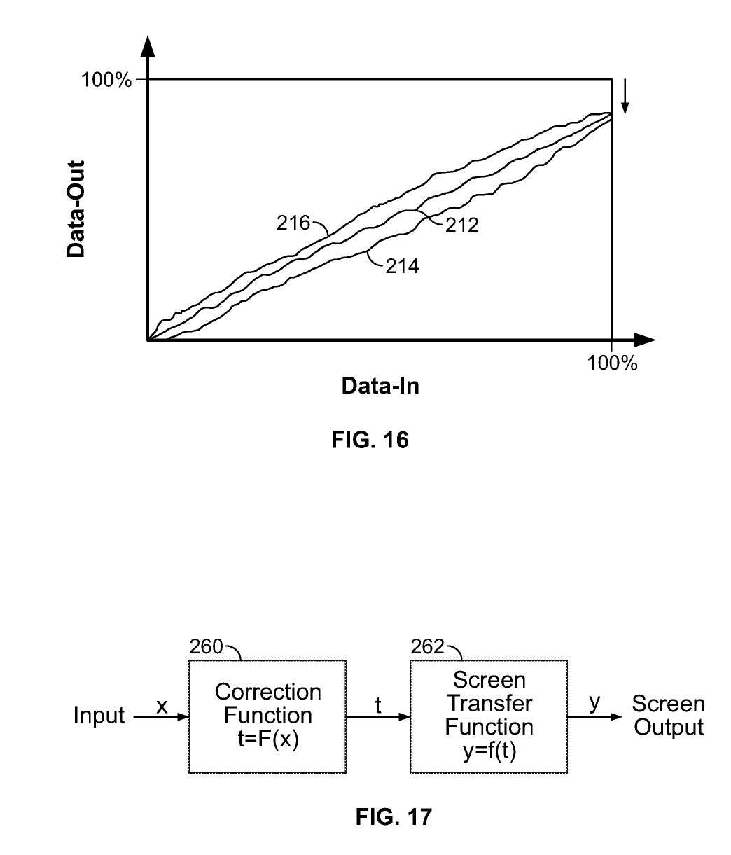

Thus, the transfer function for the display is dynamically modified, and in particular shifted and/or pivoted to compensate for the change in ambient light. It should be noted that a new compensation table may be generated based on the changed settings after a predetermined regular sampling interval (e.g., after one hour). For example, as shown in FIG. 14, a 1:1 mapping function illustrated by the curves 212, 214 and 216 may be used in a semi-dark room, which, for example, is the typical setting for an ultrasound exam room. However, if the measured light level has increased (e.g., room brighter or a highly lit room) then the lower left points of the curves 212, 214 and 216 as shown in FIG. 15 are shifted vertically upward (as illustrated by the arrow) while the upper right points of the curves 212, 214 and 216 remain fixed (e.g., the curves 212, 214 and 216 pivot about the upper right point). The amount of the shift is based on the received user input or ambient light information. If the measured light level has decreased (e.g., room darker or completely dark) then the lower left points of the curves 212, 214 and 216 as shown in FIG. 16 remain fixed (e.g., the curves 212, 214 and 216 pivot about this point) and the upper right points of the curves 212, 214 and 216 are shifted vertically downward (as illustrated by the arrow). It should be noted that various combinations of modifications to the curves may be made. For example, a combination of a shift and pivot to one or more ends of the curve may be performed. It should be noted that the tripled oscillating curves 212, 214 and 216 may be modified such that more or less curves are provided depending on system requirements, etc.

It should be noted that in some embodiments the method 220 changes the transfer function to maintain a maximum contrast. The brightness of the display is then adjusted based on the measured ambient light level (or change thereof). Color balancing also may be performed when color images are displayed such that the colors also satisfy the optimal settings or gold standard. For example, for some images, the optimal standard includes providing a slightly bluish tint to white, which may be performed by color balancing (e.g., shifting a gamma curve).

The various embodiments may be implemented in connection with any type of display. Accordingly, and for example, the screen may be optimized for viewing medical images and then returned to a normal setting for viewing other images (e.g., text or video). The optimal screen settings may be initiated by a user, for example, by selecting an optimized display view option. It should be noted that different optimized display view options with different transfer functions may be provided for viewing different types of images.

Further, the various embodiments may be integrated within a display, for example, as part of the controller for the display or may be implemented as a separate unit or module contained within or separate from the display. The various embodiments also may be implemented in hardware, software or a combination thereof.

A technical effect of at least one embodiment is automatically adjusting a display to optimize the viewing conditions. At least one ambient light detector provides ambient light level information that allows dynamic adjustment of the transfer function of the display to automatically compensate for changes in ambient light conditions to provide improved display viewing.

The various embodiments and/or components, for example, the monitor or display, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software. Further, the software may be in the form of a collection of separate programs, a program module within a larger program or a portion of a program module. The software also may include modular programming in the form of object-oriented programming. The processing of input data by the processing machine may be in response to user commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. § 112, sixth paragraph, unless and until such claim limitations expressly use the phrase “means for” followed by a statement of function void of further structure.

What is claimed is:

1. A diagnostic imaging system comprising:
an acquisition component configured to acquire image data;
a display configured to display the acquired image data;
an ambient light detector configured to detect an ambient light level; and
a display adjustment module configured to automatically adjust a display transfer function for the display based on the detected ambient light level.

2. A diagnostic imaging system in accordance with claim 1 wherein the display transfer function comprises at least one of (i) a measured real transfer function and (ii) a predetermined transfer function based on one of display type, display manufacturer and display model.

3. A diagnostic imaging system in accordance with claim 1 wherein the display transfer function defines optimal display settings.

4. A diagnostic imaging system in accordance with claim 1 wherein the display transfer function defines optimal display settings for medical images.

5. A diagnostic imaging system in accordance with claim 1 wherein the display adjustment module is configured to shift a display transfer function curve based on the detected ambient light level.

6. A diagnostic imaging system in accordance with claim 5 wherein the controller is configured to dynamically shift the display transfer function curve vertically upward if the ambient light level has increased from a previous detection and shift the display transfer function curve vertically downward if the ambient light level has decreased from a previous detection.

7. A diagnostic imaging system in accordance with claim 1 wherein the display adjustment module is configured to at least one of shift and pivot a first end of a display transfer function curve based on the detected ambient light level and at least one of shift and pivot the display transfer function curve at a second end.

8. A diagnostic imaging system in accordance with claim 1 further comprising a user interface and wherein the ambient light detector is provided in connection with the user interface.

9. A diagnostic imaging system in accordance with claim 1 wherein the ambient light detector is adjacent the display.

10. A diagnostic imaging system in accordance with claim 1 wherein the ambient light detector comprises a light guide.

11. A diagnostic imaging system in accordance with claim 10 wherein the light guide is one of angled and curved.

12. A diagnostic imaging system in accordance with claim 1 wherein the acquisition component comprises a medical imaging scanner.

13. A diagnostic imaging system in accordance with claim 12 wherein the medical imaging scanner is configured to acquire ultrasound image data.

14. A diagnostic imaging system in accordance with claim 1 further comprising a keyboard and wherein the ambient light detector is positioned on one of an edge of the keyboard and an edge of the display.

15. A diagnostic imaging system in accordance with claim 1 further comprising an adaptive filter configured to filter a detected ambient light level signal from the ambient light detector.

16. A diagnostic imaging system in accordance with claim 1 wherein the display adjustment module is configured to automatically adjust a display transfer function for the display based on a user input.

17. A medical imaging system comprising:
a display configured to display medical images;
a user interface configured to receive user inputs;
an ambient light detector configured to detect ambient light in proximity to the display; and
a display adjustment module configured to automatically adjust settings of the display based on a detected ambient light level.

18. A medical imaging system in accordance with claim 17 further comprising an ultrasound imaging probe configured to acquire ultrasound images.

19. A medical imaging system in accordance with claim 17 wherein the display adjustment module is configured to automatically adjust one of a brightness and contrast setting of the display.

20. A medical imaging system in accordance with claim 17 wherein the display adjustment module is configured to adjust the settings based on one of a measured real transfer function and a predetermined transfer function for the display that defines optimal settings.

21. A medical imaging system in accordance with claim 17 wherein the ambient light detector is provided as part of one of the display and the user interface.

22. A method for controlling a display of a diagnostic imaging system, the method comprising:
receiving ambient light level information; and
modifying a transfer function for the display based on the ambient light level information to satisfy an optimal display setting for the display.

23. A method in accordance with claim 22 wherein the modifying comprises shifting at least one end of a transfer function curve defining the transfer function.

24. A method in accordance with claim 22 wherein the modifying comprises pivoting about at least one end of a transfer function curve defining the transfer function.

25. A method in accordance with claim 22 further comprising receiving a user input and wherein the modifying is based on the received user input.
